(19) United States
(12) Patent Application Publication
(10) Pub. No.: US 2012/0094858 A1
(43) Pub. Date: Apr. 19, 2012

(54) BIOMARKERS

(75) Inventors: Sabine Bahn, Cabridge (GB); Emanuel Schwarz, Cambridge (GB); Marlis Huebner, Cambridge (GB)

(73) Assignee: Psynova Neurotech Ltd., Cambridge (GB)

(21) Appl. No.: 13/264,638

(22) PCT Filed: Apr. 16, 2010
(86) PCT No.: PCT/GB2010/050633

§ 371 Date: Dec. 20, 2011

(30) Foreign Application Priority Data
Apr. 16, 2009 (GB) 0906550.9
Dec. 21, 2009 (GB) 0922227.4

(51) Int. Cl. G01N 33/566; G01N 30/02; G01N 33/559; H01J 49/26; G01N 30/96; C40B 30/04

(52) U.S. Cl. 506/9; 436/86; 436/87; 436/161; 436/501; 204/456; 250/282; 73/61.52

(57) ABSTRACT

The invention relates to a method of diagnosing or monitoring bipolar disorders, in particular bipolar I and bipolar II disorders, such as manic psychosis.

### FIELD OF THE INVENTION

The invention relates to a method of diagnosing or monitoring bipolar disorders, in particular bipolar I and bipolar II disorders, such as manic psychosis.

### BACKGROUND OF THE INVENTION

Bipolar disorder is a psychiatric disease that describes a category of mood disorders defined by the presence of one or more episodes of abnormally elevated mood clinically referred to as mania or, if milder, hypomania. Individuals who experience manic episodes also commonly experience depressive episodes or symptoms, or mixed episodes in which features of both mania and depression are present at the same time. Such individuals also experience a decreased quality of life. These episodes are usually separated by periods of “normal” mood, but in some individuals, depression and mania may rapidly alternate, known as rapid cycling. Extreme manic episodes can sometimes lead to psychotic symptoms such as delusions and hallucinations. The disorder has been subdivided into bipolar I, bipolar II, cyclothymia, and other types, based on the nature and severity of mood episodes experienced; the range is often described as the bipolar spectrum.

Bipolar I disorder is characterised by manic episodes; the “high” of the manic-depressive cycle. Generally, this manic period is followed by a period of depression, although some bipolar I individuals may not experience a major depressive episode. Mixed states, where both manic or hypomanic symptoms and depressive symptoms occur at the same time, also occur frequently with bipolar I patients (for example, depression with the racing thoughts of mania). Also, dysphoric mania is common and is mania characterised by anger and irritability.

Bipolar II disorder is characterised by major depressive episodes alternating with episodes of hypomania, a milder form of mania. Hypomanic episodes can be a less disruptive form of mania and may be characterised by low-level, non-psychotic symptoms of mania, such as increased energy or a more elevated mood than usual. It may not affect an individual's ability to function on a day to day basis. The criteria for hypomania differ from those for mania only by their shorter duration (at least 4 days instead of 1 week) and milder severity (no marked impairment of functioning, hospitalisation or psychotic features).

If the depressive and manic symptoms last for two years and do not meet the criteria for a major depressive or a manic episode then the diagnosis is classified as a cyclothymic disorder, which is a less severe form of bipolar affective disorder. Cyclothymic disorder is diagnosed over the course of two years and is characterised by frequent short periods of hypomania and depressive symptoms separated by periods of stability.

Rapid cycling occurs when an individual's mood fluctuates from depression to hypomania or mania in rapid succession with little or no periods of stability in between. One is said to experience rapid cycling when one has had four or more episodes in a given year that meet criteria for major depressive, manic, mixed or hypomanic episodes. Some people who rapid cycle can experience monthly, weekly or even daily shifts in polarity (sometimes called ultra rapid cycling).

To date, no empirical diagnostic tests are available, making diagnosis a subjective evaluation which often leads to misdiagnosis and delay in accurate treatment. When symptoms of mania, depression, mixed mood or hypomania are caused directly by a medical disorder, such as thyroid disease or a stroke, the current diagnosis is Mood Disorder Due to a General Medical Condition.

In a manic mood brought about through an antidepressant, ECT or through an individual using street drugs, the diagnosis is Substance-Induced Mood Disorder, with Manic Features.

Diagnosis of bipolar disorders has been used to categorise manic episodes which occur as a result of taking an antidepressant medication, rather than occurring spontaneously. Confusingly, it has also been used in instances where an individual experiences hypomania or cyclothymia (i.e. less severe mania) without major depression.

### SUMMARY OF THE INVENTION

According to a first aspect of the invention, there is provided the use of one or more first analytes selected from: Cancer Antigen 125, HCC-4, Apolipoprotein B, IL-11, CD5L, IL-6 receptor, Kidney injury molecule 1 (KIM-1), MMP-2,

Transferrin, Testosterone, C. jejuni, T. cruzi and Glucagon, as a biomarker for bipolar I or bipolar II disorders, or predisposition thereto.

According to a second aspect of the invention, there is provided the use of two or more second analytes selected from: Eotaxin-3, LH (Luteinizing Hormone), Histone H2b Antibody, Apolipoprotein CIII, Histone H4 Antibody, Fas Ligand, IgA, C. trachomatis, IL-13, Histone H1 Antibody, CTGF (Connective Tissue Growth Factor), CD40 Ligand, EGF, Stem Cell Factor, Lymphotactin, Myeloperoxidase, TSP 1, IL-16, MIP-1 beta, Apolipoprotein A2, Apolipoprotein CI, IL-17, Thrombopoietin, Calbindin, EGF receptor, Follicle Stimulating Hormone (FSH), ANG 2 (Angiopoietin 2), IL-7, MCP-2, Peptide YY (PYY), Thyroid Stimulating Hormone (TSH; beta subunit), Vitronectin, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas, as a biomarker for bipolar I or bipolar II disorders, or predisposition thereto.

According to a third aspect of the invention, there is provided a method of diagnosing or monitoring bipolar I or bipolar II disorders, or predisposition thereto, comprising detecting and/or quantifying, in a sample from a test subject, the analyte biomarkers defined herein.

According to a fourth aspect of the invention, there is provided a method of diagnosing bipolar I or bipolar II disorders, or predisposition in an individual thereto, comprising:
(a) obtaining a biological sample from an individual;
(b) quantifying the amounts of the analyte biomarkers as defined herein;
(c) comparing the amounts of the analyte biomarkers in the biological sample with the amounts present in a normal control biological sample from a normal subject, such that a difference in the level of the analyte biomarkers in the biological sample is indicative of bipolar I or bipolar II disorders, or predisposition thereto.

According to a fifth aspect of the invention, there is provided a method of monitoring efficacy of a therapy in a subject having, suspected of having, or of being predisposed to bipolar I or bipolar II disorders, comprising detecting and/or quantifying, in a sample from said subject, the analyte biomarkers defined herein.

According to a sixth aspect of the invention, there is provided a method of determining the efficacy of therapy for bipolar I or bipolar II disorders in an individual subject comprising:
(a) obtaining a biological sample from an individual;
(b) quantifying the amounts of the analyte biomarkers as defined herein;
(c) comparing the amounts of the analyte biomarkers in the biological sample with the amounts present in a sample obtained from the individual on a previous occasion, such that a difference in the level of the analyte biomarkers in the biological sample is indicative of a beneficial effect of the therapy.

A further aspect of the invention provides ligands, such as naturally occurring or chemically synthesised compounds, capable of specific binding to the analyte biomarker. A ligand according to the invention may comprise a peptide, an antibody or a fragment thereof, or an aptamer or oligonucleotide, capable of specific binding to the analyte biomarker. The antibody can be a monoclonal antibody or a fragment thereof capable of specific binding to the analyte biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

A biosensor according to the invention may comprise the analyte biomarker or a structural/shape mimic thereof capable of specific binding to an antibody against the analyte biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or the use of a biosensor of the invention, or an array of the invention, or a kit of the invention to detect and/or quantify the analyte. In these uses, the detection and/or quantification can be performed on a biological sample such as from the group consisting of CSF, whole blood, blood serum, plasma, urine, saliva, or other bodily fluid, breath, e.g. as condensed breath, or an extract or purification therefrom, or dilution thereof.

Diagnostic or monitoring kits are provided for performing methods of the invention. Such kits will suitably comprise a ligand according to the invention, for detection and/or quantification of the analyte biomarker, and/or a biosensor, and/or an array as described herein, optionally together with instructions for use of the kit.

A further aspect of the invention is a kit for monitoring or diagnosing bipolar I or bipolar II disorders, comprising a biosensor capable of detecting and/or quantifying one or more of the first analyte biomarkers as defined herein.

A further aspect of the invention is a kit for monitoring or diagnosing bipolar I or bipolar II disorders, comprising a biosensor capable of detecting and/or quantifying two or more of the second analyte biomarkers as defined herein.

Biomarkers for bipolar I or bipolar II disorders are essential targets for discovery of novel targets and drug molecules that retard or halt progression of the disorder. As the level of the analyte biomarker is indicative of disorder and of drug response, the biomarker is useful for identification of novel therapeutic compounds in in vitro and/or in vivo assays. Biomarkers of the invention can be employed in methods for screening for compounds that modulate the activity of the analyte.

Thus, in a further aspect of the invention, there is provided the use of a ligand, as described, which can be a peptide, antibody or fragment thereof or aptamer or oligonucleotide according to the invention; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the analyte in a subject, comprising administering a test substance to a subject animal and detecting and/or quantifying the level of the analyte biomarker present in a test sample from the subject.

### DETAILED DESCRIPTION OF THE INVENTION

According to a first aspect of the invention, there is provided the use of one or more first analytes selected from: Cancer Antigen 125, HCC-4, Apolipoprotein B, IL-11, CD5L, IL-6 receptor, Kidney injury molecule 1 (KIM-1), MMP-2, Transferrin, Testosterone, C. jejuni, T. cruzi and Glucagon, as a biomarker for bipolar I or bipolar II disorders, or predisposition thereto.

In one embodiment, the first analyte is selected from Cancer Antigen 125. Data is provided herein which demonstrates that Cancer Antigen 125 provided a statistically significant marker in studies conducted with both samples from bipolar patients as well as patients with the manic psychosis episode of bipolar disorder (as shown in Examples 2 and 3). Thus, according to a further aspect of the invention, there is provided the use of Cancer Antigen 125 as a biomarker for bipolar I or bipolar II disorders, or predisposition thereto. In one embodiment of this aspect of the invention, the use additionally comprises one or more analytes selected from HCC-4, Apolipoprotein B, IL-11, CD5L, IL-6 receptor, Kidney injury molecule 1 (KIM-1), MMP-2, Transferrin, Eotaxin-3, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni, T. cruzi, Myeloperoxidase, TSP 1, IL-16, MIP-1 beta, Glucagon, Apolipoprotein A2, Apolipoprotein CI, IL-17, Thrombopoietin, Calbindin, EGF receptor, Follicle Stimulating Hormone (FSH), IL-7, MCP-2, Peptide YY (PYY), Thyroid Stimulating Hormone (TSH; beta subunit), Vitronectin, ANG 2 (Angiopoietin 2), Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas.

In one embodiment of the first aspect of the invention, the use additionally comprises the use of one or more second analytes selected from: Eotaxin-3, LH (Luteinizing Hormone), Histone H2b Antibody, Apolipoprotein CIII, Histone H4 Antibody, Fas Ligand, IgA, C. trachomatis, IL-13, Histone H1 Antibody, CTGF (Connective Tissue Growth Factor), CD40 Ligand, EGF, Stem Cell Factor, Lymphotactin, Myeloperoxidase, TSP 1, IL-16, MIP-1 beta, Apolipoprotein A2, Apolipoprotein CI, IL-17, Thrombopoietin, Calbindin, EGF receptor, Follicle Stimulating Hormone (FSH), ANG 2 (Angiopoietin 2), IL-7, MCP-2, Peptide YY (PYY), Thyroid Stimulating Hormone (TSH; beta subunit), Vitronectin, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas.

According to a second aspect of the invention, there is provided the use of two or more second analytes selected from: Eotaxin-3, LH (Luteinizing Hormone), Histone H2b Antibody, Apolipoprotein CIII, Histone H4 Antibody, Fas Ligand, IgA, C. trachomatis, IL-13, Histone H1 Antibody, CTGF (Connective Tissue Growth Factor), CD40 Ligand, EGF, Stem Cell Factor, Lymphotactin, Myeloperoxidase, TSP 1, IL-16, MIP-1 beta, Apolipoprotein A2, Apolipoprotein CI, IL-17, Thrombopoietin, Calbindin, EGF receptor, Follicle Stimulating Hormone (FSH), ANG 2 (Angiopoietin 2), IL-7, MCP-2, Peptide YY (PYY), Thyroid Stimulating Hormone (TSH; beta subunit), Vitronectin, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas, as a biomarker for bipolar I or bipolar II disorders, or predisposition thereto.

The term “biomarker” means a distinctive biological or biologically derived indicator of a process, event, or condition. Analyte biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment.

It will be readily apparent to the skilled person that the first and second analytes listed herein are known and have been described in the literature, however, for completeness, full characterising information for these analytes is provided in Table 1:

TABLE 1
Characterising Information of the First and Second Analytes of
the Invention

| | Accession |
|---|---|
| Analyte | Number |
| Endothelin 1 | P80511 |
| Eotaxin-3 | Q9Y258 |
| MIF | P14174 |
| HCC 4 | O15467 |
| LH (Luteinizing Hormone) | P01229 |
| Histone Antibody | N/A |
| Histone H2b Antibody | N/A |
| TNF alpha | P01229 |
| Apolipoprotein CIII | P02656 |
| IGF BP 2 | P18065 |
| Histone H4 Antibody | N/A |
| Progesterone | N/A |
| Anti Nuclear Antibody | N/A |
| Testosterone | N/A |
| Fas Ligand | Q0VHD7 |
| IgA | N/A |
| C. trachomatis | N/A |
| IgM | N/A |
| IL-13 | P35225 |
| Histone H1 Antibody | N/A |
| CTGF (Connective Tissue Growth Factor) | P29279 |
| Apolipoprotein A1 | P02647 |
| CD40 Ligand | P29965 |
| EGF | P01133 |
| CD40 | P25942 |
| Stem Cell Factor | P21583 |
| Lymphotactin | P47992 |
| C. jejuni | N/A |
| T. cruzi | N/A |
| GST | P09210 |
| Alpha 2 Macroglobulin | P01023 |
| ANG 2 Angiopoietin 2 | O15123 |
| Thrombopoietin | P40225 |
| Myeloperoxidase | P05164 |
| IL-8 | P10145 |
| TSP 1 | P07996 |
| IL-16 | Q14005 |
| FSH (Follicle Stimulating Hormone) | P01225 |
| MIP 1 beta | P13236 |
| SOD | P08294 |
| Fas | P25445 |
| Glucagon | P01275 |
| Apolipoprotein B | N/A |
| IL-11 | N/A |
| Cancer Antigen 125 | N/A |
| CD5L | N/A |
| IL-6 receptor | N/A |
| Kidney injury molecule 1 (KIM-1) | N/A |
| MMP-2 | N/A |
| Transferrin | N/A |
| Apolipoprotein A2 | N/A |
| Apolipoprotein CI | N/A |
| IL-17 | N/A |
| Calbindin | N/A |
| EGF receptor | N/A |
| IL-7 | N/A |
| MCP-2 | N/A |
| Peptide YY (PYY) | N/A |
| Thyroid Stimulating Hormone (TSH; beta subunit) | N/A |
| Vitronectin | N/A |

According to one particular aspect of the invention, there is provided the use of one or more first analytes selected from: Eotaxin-3, HCC-4, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni, T. cruzi, ANG 2 (Angiopoietin 2), Thrombopoietin, Myeloperoxidase, TSP 1, IL-16, FSH (Follicle Stimulating Hormone), MIP-1 beta and Glucagon, as a biomarker for bipolar I or bipolar II disorders, or predisposition thereto.

In one embodiment, the one or more first analytes are selected from: Eotaxin-3, HCC-4, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni, T. cruzi, Myeloperoxidase, TSP 1, IL-16, MIP-1 beta and Glucagon, as a biomarker for bipolar I or bipolar II disorders, or predisposition thereto.

In one embodiment, the first analyte is other than Histone H2b Antibody. In one embodiment, the first analyte is other than Histone H4 Antibody. In one embodiment, the first analyte is other than Histone H1 Antibody. In one embodiment, the first analyte is other than MIP 1 beta. In one embodiment, the first analyte is other than Glucagon. In one embodiment, the first analyte is other than MMP-2. In one embodiment, the first analyte is other than transferrin.

In one embodiment, the first analyte is selected from: Apolipoprotein B, IL-11, Cancer Antigen 125, CD5L, IL-6 receptor, Kidney injury molecule 1 (KIM-1), Eotaxin-3, HCC-4, LH (Luteinizing Hormone), Apolipoprotein CIII, Testosterone, Fas Ligand, IgA, C. trachomatis, IL-13, CTGF (Connective Tissue Growth Factor), CD40 Ligand, EGF, Stem Cell Factor, Lymphotactin, C. jejuni, T. cruzi, Myeloperoxidase, TSP 1 and IL-16.

In one embodiment, the first analyte is selected from: Eotaxin-3, HCC-4, LH (Luteinizing Hormone), Apolipoprotein CIII, Testosterone, Fas Ligand, IgA, C. trachomatis, IL-13, CTGF (Connective Tissue Growth Factor), CD40 Ligand, EGF, Stem Cell Factor, Lymphotactin, C. jejuni, T. cruzi, ANG 2 (Angiopoietin 2), Thrombopoietin, Myeloperoxidase, TSP 1, IL-16 and FSH (Follicle Stimulating Hormone).

In one embodiment, the first analyte is selected from: Eotaxin-3, HCC-4, LH (Luteinizing Hormone), Apolipoprotein CIII, Testosterone, Fas Ligand, IgA, C. trachomatis, IL-13, CTGF (Connective Tissue Growth Factor), CD40 Ligand, EGF, Stem Cell Factor, Lymphotactin, C. jejuni, T. cruzi, Myeloperoxidase, TSP 1 and IL-16.

In one embodiment, the first analyte is selected from: Eotaxin-3, HCC-4, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni and T. cruzi.

In one embodiment, the first analyte is selected from: Eotaxin-3, HCC-4, LH (Luteinizing Hormone), Apolipoprotein CIII, Testosterone, Fas Ligand, IgA, C. trachomatis, IL-13, CTGF (Connective Tissue Growth Factor), CD40 Ligand, EGF, Stem Cell Factor, Lymphotactin, C. jejuni and T. cruzi.

In one embodiment, the first analyte is selected from: Apolipoprotein B, IL-11, Cancer Antigen 125, CD5L, IL-6 receptor, Kidney injury molecule 1 (KIM-1), MMP-2 and Transferrin.

In a further embodiment, the first analyte is selected from: Cancer Antigen 125, CD5L, IL-6 receptor, MMP-2 and Transferrin.

In a further embodiment, the first analyte is selected from: IL-6 receptor or MMP-2.

In a further embodiment, the first analyte is selected from: Cancer Antigen 125, CD5L and Transferrin.

According to a further particular aspect of the invention, there is provided the use of two or more second analytes selected from: Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas, as a biomarker for bipolar I or bipolar II disorders, or predisposition thereto.

In one embodiment, the use additionally comprises the use of one or more second analytes selected from: Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD, Fas, Thrombopoietin, Follicle Stimulating Hormone (FSH) and ANG 2 (Angiopoietin 2).

In one embodiment, the use additionally comprises the use of one or more second analytes selected from: Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas.

In one embodiment, the one or more second analytes additionally comprise Histone H2b Antibody. In one embodiment, the one or more second analytes additionally comprise Histone H4 Antibody. In one embodiment, the one or more second analytes additionally comprise Histone H1 Antibody. In one embodiment, the one or more second analytes additionally comprise MIP 1 beta. In one embodiment, the one or more second analytes additionally comprise Glucagon. In one embodiment, the one or more second analytes additionally comprise MMP-2. In one embodiment, the one or more second analytes additionally comprise Transferrin.

According to a further aspect of the invention, there is provided the use of two or more second analytes selected from: Apolipoprotein A2, Apolipoprotein CI, IL-17, Thrombopoietin, Calbindin, EGF receptor, Follicle Stimulating Hormone (FSH), ANG 2 (Angiopoietin 2), IL-7, MCP-2, Peptide YY (PYY), Thyroid Stimulating Hormone (TSH; beta subunit), Vitronectin, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD, Fas, Histone H2b Antibody, Histone H4 Antibody, Histone H1 Antibody, MIP 1 beta, Glucagon, MMP-2 and Transferrin as a biomarker for bipolar I or bipolar II disorders, or predisposition thereto.

According to a further particular aspect of the invention, there is provided the use of two or more second analytes selected from: Endothelin 1, MIF, Histone Antibody, Histone H2b Antibody, TNF alpha, IGF BP 2, Histone H4 Antibody, Progesterone, Anti Nuclear Antibody, IgM, Histone H1 Antibody, Apolipoprotein A1, CD40, GST, Alpha 2 Macroglobulin, IL-8, MIP 1 beta, SOD, Fas and Glucagon, as a biomarker for bipolar I or bipolar II disorders, or predisposition thereto.

In one embodiment, the second analyte is selected from: Endothelin 1, MIF,

Histone Antibody, TNF alpha, IGF BP 2, Progesterone, Anti Nuclear Antibody, IgM, Apolipoprotein A1, CD40 and GST.

In one embodiment, the second analyte is selected from: Apolipoprotein A2, Apolipoprotein CI, IL-17, Thrombopoietin, Calbindin, EGF receptor, Follicle Stimulating Hormone (FSH), IgM, IL-7, MCP-2, Peptide YY (PYY), Thyroid Stimulating Hormone (TSH; beta subunit) and Vitronectin.

In one embodiment, the second analyte is selected from: Apolipoprotein A2, IL-17, Thrombopoietin, Calbindin, Follicle Stimulating Hormone (FSH), IL-7, MCP-2 and Peptide YY (PYY).

In a further embodiment, the second analyte is selected from: Apolipoprotein A2, IL-17, Calbindin, Follicle Stimulating Hormone (FSH) and MCP-2.

In a further embodiment, the second analyte is selected from: Thrombopoietin, IL-7 and Peptide YY (PYY).

According to a further aspect of the invention, there is provided the use of Cancer Antigen 125, Apolipoprotein A2, Apolipoprotein B, Apolipoprotein CI, IL-11, IL-17, Thrombopoietin, Calbindin, CD5L, EGF receptor, Follicle Stimulating Hormone (FSH), IgM, IL-6 receptor, IL-7, Kidney injury molecule 1 (KIM-1), MCP-2, MMP-2, Peptide YY (PYY), Thyroid Stimulating Hormone (TSH; beta subunit), Transferrin and Vitronectin as a specific panel of analyte biomarkers for bipolar I or bipolar II disorders, or predisposition thereto. Data is presented in Example 2 herein which demonstrates that the above mentioned 21 analytes were found to be significantly altered in pre-symptomatic bipolar disorder patients compared to non-symptomatic healthy controls. Therefore, this specific panel of 21 biomarkers provided by this aspect of the invention is a sensitive and specific predictor for the presence of bipolar disorder.

According to a further aspect of the invention, there is provided the use of Eotaxin-3, HCC-4, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni, T. cruzi, ANG 2 (Angiopoietin 2), Thrombopoietin, Myeloperoxidase, TSP 1, IL-16, FSH (Follicle Stimulating Hormone), MIP-1 beta, Glucagon, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas as a specific panel of analyte biomarkers for bipolar I or bipolar II disorders, or predisposition thereto. Data is presented in Example 1 herein which demonstrates that the above mentioned 42 analytes were found to be significantly altered between bipolar disorder and the control group. Therefore, this specific panel of 42 biomarkers provided by the invention is a sensitive and specific predictor for the presence of bipolar disease.

According to a further aspect of the invention, there is provided the use of Cancer Antigen 125, Eotaxin-3, HCC-4, LH (Luteinizing Hormone), Histone H2b Antibody, Apolipoprotein CIII, Histone H4 Antibody, Testosterone, Fas Ligand, IgA, C. trachomatis, IL-13, Histone H1 Antibody, CTGF (Connective Tissue Growth Factor), CD40 Ligand, EGF, Stem Cell Factor, Lymphotactin, C. jejuni, T. cruzi, ANG 2 (Angiopoietin 2), Thrombopoietin, Myeloperoxidase, TSP 1, IL-16, FSH (Follicle Stimulating Hormone), MIP-1 beta, Glucagon, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD, Fas, Apolipoprotein A2, Apolipoprotein B, Apolipoprotein CI, IL-11, IL-17, Calbindin, CD5L, EGF receptor, IL-6 receptor, IL-7, Kidney injury molecule 1 (KIM-1), MCP-2, MMP-2, Peptide YY (PYY), Thyroid Stimulating Hormone (TSH; beta subunit), Transferrin and Vitronectin as a specific panel of analyte biomarkers for bipolar I or bipolar II disorders, or predisposition thereto. Data is presented in Tables 3 and 5 herein which demonstrates that the above mentioned 60 analytes were found to be significantly altered in bipolar disorder patients compared to healthy controls. Therefore, this specific panel of 60 biomarkers provided by this aspect of the invention is a sensitive and specific predictor for the presence of bipolar disorder. In particular, it can be noted that the biomarkers with a fold change of <1 are those which are decreased in patients with bipolar disorder. By contrast, the biomarkers with a fold change of >1 are those which are increased in patients with bipolar disorder.

For example, it can be noted that the levels of the following biomarkers increased in patients with bipolar disorder: HCC-4, LH (Luteinizing Hormone), Histone H2b Antibody, Testosterone, Fas Ligand, C. trachomatis, Histone H1 Antibody, CTGF (Connective Tissue Growth Factor), CD40 Ligand, EGF, Lymphotactin, C. jejuni, T. cruzi, ANG 2 (Angiopoietin 2), Myeloperoxidase, TSP 1, IL-16, FSH (Follicle Stimulating Hormone), MIP-1 beta, Endothelin 1, MIF, Histone Antibody, IGF BP 2, Progesterone, Anti Nuclear Antibody, CD40, GST, IL-8, SOD, Fas, Apolipoprotein A2, Apolipoprotein CI, IL-17, Calbindin, EGF receptor, IL-6 receptor, Kidney injury molecule 1 (KIM-1), MCP-2 and MMP-2.

Furthermore, it can be noted that the levels of the following biomarkers decreased in patients with bipolar disorder: Cancer Antigen 125, Eotaxin-3, Apolipoprotein CIII, Histone H4 Antibody, IgA, IL-13, Stem Cell Factor, Thrombopoietin, TNF alpha, Apolipoprotein A1, Alpha 2 Macroglobulin, Apolipoprotein B, IL-11, CD5L, Follicle Stimulating Hormone (FSH), IL-7, Peptide YY (PYY), Thyroid Stimulating Hormone (TSH; beta subunit), Transferrin and Vitronectin.

According to a further aspect of the invention, there is provided the use of HCC-4, LH (Luteinizing Hormone), Histone H2b Antibody, Testosterone, Fas Ligand, C. trachomatis, Histone H1 Antibody, CTGF (Connective Tissue Growth Factor), CD40 Ligand, EGF, Lymphotactin, C. jejuni, T. cruzi, ANG 2 (Angiopoietin 2), Myeloperoxidase, TSP 1, IL-16, FSH (Follicle Stimulating Hormone), MIP-1 beta, Endothelin 1, MIF, Histone Antibody, IGF BP 2, Progesterone, Anti Nuclear Antibody, CD40, GST, IL-8, SOD, Fas, Apolipoprotein A2, Apolipoprotein CI, IL-17, Calbindin, EGF receptor, IL-6 receptor, Kidney injury molecule 1 (KIM-1), MCP-2 and MMP-2 as a specific panel of analyte biomarkers for bipolar I or bipolar II disorders, or predisposition thereto.

According to a further aspect of the invention, there is provided a method of diagnosing bipolar I or bipolar II disorders, or predisposition thereto, in an individual thereto comprising
a) obtaining a biological sample from an individual;
b) quantifying the amounts of a panel of analyte biomarkers in the biological sample, wherein the panel of analyte biomarkers comprises HCC-4, LH (Luteinizing Hormone), Histone H2b Antibody, Testosterone, Fas Ligand, C. trachomatis, Histone H1 Antibody, CTGF (Connective Tissue Growth Factor), CD40 Ligand, EGF, Lymphotactin, C. jejuni, T. cruzi, ANG 2 (Angiopoietin 2), Myeloperoxidase, TSP 1, IL-16, FSH (Follicle Stimulating Hormone), MIP-1 beta, Endothelin 1, MIF, Histone Antibody, IGF BP 2, Progesterone, Anti Nuclear Antibody, CD40, GST, IL-8, SOD, Fas, Apolipoprotein A2, Apolipoprotein CI, IL-17, Calbindin, EGF receptor, IL-6 receptor, Kidney injury molecule 1 (KIM-1), MCP-2 and MMP-2; and
c) comparing the amounts of the panel of analyte biomarkers in the biological sample with the amounts present in a normal control biological sample from a normal subject, wherein a higher level of the panel of analyte biomarkers in the biological sample is indicative of bipolar I or bipolar II disorders, or predisposition thereto.

In one embodiment, the higher level is a >1 fold difference relative to the control sample, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15 or 20 or any ranges therebetween. In one embodiment, the higher level is between 1 and 15 fold difference relative to the control sample, such as between 1.5 and 10.

According to a further aspect of the invention, there is provided the use of Cancer Antigen 125, Eotaxin-3, Apolipoprotein CIII, Histone H4 Antibody, IgA, IL-13, Stem Cell Factor, Thrombopoietin, TNF alpha, Apolipoprotein A1, Alpha 2 Macroglobulin, Apolipoprotein B, IL-11, CD5L, Follicle Stimulating Hormone (FSH), IL-7, Peptide YY (PYY), Thyroid Stimulating Hormone (TSH; beta subunit), Transferrin and Vitronectin as a specific panel of analyte biomarkers for bipolar I or bipolar II disorders, or predisposition thereto.

According to a further aspect of the invention, there is provided a method of diagnosing bipolar I or bipolar II disorders, or predisposition thereto, in an individual thereto comprising
a) obtaining a biological sample from an individual;
b) quantifying the amounts of a panel of analyte biomarkers in the biological sample, wherein the panel of analyte biomarkers comprises Cancer Antigen 125, Eotaxin-3, Apolipoprotein CIII, Histone H4 Antibody, IgA, IL-13, Stem Cell Factor, Thrombopoietin, TNF alpha, Apolipoprotein A1, Alpha 2 Macroglobulin, Apolipoprotein B, IL-11, CD5L, Follicle Stimulating Hormone (FSH), IL-7, Peptide YY (PYY), Thyroid Stimulating Hormone (TSH; beta subunit), Transferrin and Vitronectin; and
c) comparing the amounts of the panel of analyte biomarkers in the biological sample with the amounts present in a normal control biological sample from a normal subject, wherein a lower level of the panel of analyte biomarkers in the biological sample is indicative of bipolar I or bipolar II disorders, or predisposition thereto.

In one embodiment, the lower level is a <1 fold difference relative to the control sample, such as a fold difference of 0.9, 0.8, 0.7, 0.6, 0.5, 0.4, 0.3, 0.2, 0.1, 0.05, 0.01 or any ranges therebetween. In one embodiment, the lower level is between 0.1 and 0.95 fold difference relative to the control sample, such as between 0.2 and 0.95.

According to a further aspect of the invention, there is provided the use of Cancer Antigen 125, MIF, TNF-alpha, Progesterone, CD40 Ligand, EGF, CD40, Alpha-2 Macroglobulin, Thrombopoietin, Myeloperoxidase, IL-16, MIP-lbeta, FAS and Calbindin as a specific panel of analyte biomarkers for bipolar I or bipolar II disorders, or predisposition thereto, such as the manic psychosis episode of bipolar disorder.

Data is presented in Table 7 herein which demonstrates that the above mentioned 14 analytes were found to be significantly altered in bipolar disorder patients with a manic psychosis episode compared to healthy controls. Therefore, this specific panel of 14 biomarkers provided by this aspect of the invention is a sensitive and specific predictor for the presence of the manic psychosis episode of bipolar disorder. In particular, it can be noted that the biomarkers with a fold change of <1 are those which are decreased in patients with the manic psychosis episode of bipolar disorder. By contrast, the biomarkers with a fold change of >1 are those which are increased in patients with the manic psychosis episode of bipolar disorder.

For example, it can be noted that the levels of all listed biomarkers increased in patients with the manic psychosis episode of bipolar disorder; i.e.: Cancer Antigen 125, MIF, TNF-alpha, Progesterone, CD40 Ligand, EGF, CD40, Alpha-2 Macroglobulin, Thrombopoietin, Myeloperoxidase, IL-16, MIP-1beta, FAS and Calbindin.

According to a further aspect of the invention, there is provided the use of Cancer Antigen 125, MIF, TNF-alpha, Progesterone, CD40 Ligand, EGF, CD40, Alpha-2 Macroglobulin, Thrombopoietin, Myeloperoxidase, IL-16, MIP-1beta, FAS and Calbindin as a specific panel of analyte biomarkers for the manic psychosis episode of bipolar disorder, or predisposition thereto.

According to a further aspect of the invention, there is provided a method of diagnosing the manic psychosis episode of bipolar disorder, or predisposition thereto, in an individual thereto comprising
a) obtaining a biological sample from an individual;
b) quantifying the amounts of a panel of analyte biomarkers in the biological sample, wherein the panel of analyte biomarkers comprises Cancer Antigen 125, MIF, TNF-alpha, Progesterone, CD40 Ligand, EGF, CD40, Alpha-2 Macroglobulin, Thrombopoietin, Myeloperoxidase, IL-16, MIP-lbeta, FAS and Calbindin; and
c) comparing the amounts of the panel of analyte biomarkers in the biological sample with the amounts present in a normal control biological sample from a normal subject, wherein a higher level of the panel of analyte biomarkers in the biological sample is indicative of the manic psychosis episode of bipolar disorder, or predisposition thereto.

In one embodiment, the higher level is a >1 fold difference relative to the control sample, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15, 20, 25, 30, 35, 40, 45, 50, 55, 60, 65, 70, 75, 80, 85, 90, 95, 100 or any ranges therebetween. In one embodiment, the higher level is between 1 and 15 fold difference relative to the control sample, such as between 1.2 and 10.

In one embodiment, one or more of the biomarkers may be replaced by a molecule, or a measurable fragment of the molecule, found upstream or downstream of the biomarker in a biological pathway.

As used herein, the term “biosensor” means anything capable of detecting the presence of the biomarker. Examples of biosensors are described herein.

Biosensors according to the invention may comprise a ligand or ligands, as described herein, capable of specific binding to the analyte biomarker. Such biosensors are useful in detecting and/or quantifying an analyte of the invention.

Diagnostic kits for the diagnosis and monitoring of bipolar I or bipolar II disorders are described herein. In one embodiment, the kits additionally contain a biosensor capable of detecting and/or quantifying an analyte biomarker.

Monitoring methods of the invention can be used to monitor onset, progression, stabilisation, amelioration and/or remission.

In methods of diagnosing or monitoring according to the invention, detecting and/or quantifying the analyte biomarker in a biological sample from a test subject may be performed on two or more occasions. Comparisons may be made between the level of biomarker in samples taken on two or more occasions. Assessment of any change in the level of the analyte biomarker in samples taken on two or more occasions may be performed. Modulation of the analyte biomarker level is useful as an indicator of the state of the bipolar I or bipolar II disorders or predisposition thereto. An increase in the level of the biomarker, over time is indicative of onset or progression, i.e. worsening of this disorder, whereas a decrease in the level of the analyte biomarker indicates amelioration or remission of the disorder, or vice versa.

A method of diagnosis or monitoring according to the invention may comprise quantifying the analyte biomarker in a test biological sample from a test subject and comparing the level of the analyte present in said test sample with one or more controls.

The control used in a method of the invention can be one or more control(s) selected from the group consisting of: the level of biomarker analyte found in a normal control sample from a normal subject, a normal biomarker analyte level; a normal biomarker analyte range, the level in a sample from a subject with bipolar I or bipolar II disorders, or a diagnosed predisposition thereto; bipolar I or bipolar II disorders biomarker analyte level, or bipolar I or bipolar II disorders biomarker analyte range.

In one embodiment, there is provided a method of diagnosing bipolar I or bipolar II disorders, or predisposition thereto, which comprises:
(a) quantifying the amount of the analyte biomarker in a test biological sample; and
(b) comparing the amount of said analyte in said test sample with the amount present in a normal control biological sample from a normal subject.

For biomarkers which are increased in patients with bipolar I or bipolar II disorders, a higher level of the peptide biomarker in the test sample relative to the level in the normal control is indicative of the presence of bipolar I or bipolar II disorders, or predisposition thereto; an equivalent or lower level of the peptide in the test sample relative to the normal control is indicative of absence of bipolar I or bipolar II disorders and/or absence of a predisposition thereto. For biomarkers which are decreased in patients with bipolar I or bipolar II disorders, a lower level of the peptide biomarker in the test sample relative to the level in the normal control is indicative of the presence of bipolar I or bipolar II disorders, or predisposition thereto; an equivalent or lower level of the peptide in the test sample relative to the normal control is indicative of absence of bipolar I or bipolar II disorders and/or absence of a predisposition thereto.

The term “diagnosis” as used herein encompasses identification, confirmation, and/or characterisation of bipolar I or bipolar II disorders, or predisposition thereto. By predisposition it is meant that a subject does not currently present with the disorder, but is liable to be affected by the disorder in time. Methods of monitoring and of diagnosis according to the invention are useful to confirm the existence of a disorder, or predisposition thereto; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects), reducing “down-time” and relapse rates.

Also provided is a method of monitoring efficacy of a therapy for bipolar I or bipolar II disorders in a subject having such a disorder, suspected of having such a disorder, or of being predisposed thereto, comprising detecting and/or quantifying the analyte present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for bipolar I or bipolar II disorders in a subject, comprising:
(a) quantifying the amount of the analyte biomarker; and
(b) comparing the amount of said analyte in said test sample with the amount present in one or more control(s) and/or one or more previous test sample(s) taken at an earlier time from the same test subject.

For biomarkers which are increased in patients with bipolar I or bipolar II disorders, a decrease in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto. For biomarkers which are decreased in patients with bipolar I or bipolar II disorders, an increase in the level of the peptide biomarker in the test sample relative to the level in a previous test sample taken earlier from the same test subject is indicative of a beneficial effect, e.g. stabilisation or improvement, of said therapy on the disorder, suspected disorder or predisposition thereto.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 or 12 months. Samples may be taken prior to and/or during and/or following an anti-depressant therapy. Samples can be taken at intervals over the remaining life, or a part thereof, of a subject.

The term “detecting” as used herein means confirming the presence of the analyte biomarker present in the sample. Quantifying the amount of the biomarker present in a sample may include determining the concentration of the analyte biomarker present in the sample. Detecting and/or quantifying may be performed directly on the sample, or indirectly on an extract therefrom, or on a dilution thereof.

In alternative aspects of the invention, the presence of the analyte biomarker is assessed by detecting and/or quantifying antibody or fragments thereof capable of specific binding to the biomarker that are generated by the subject's body in response to the analyte and thus are present in a biological sample from a subject having bipolar I or bipolar II disorders or a predisposition thereto.

Detecting and/or quantifying can be performed by any method suitable to identify the presence and/or amount of a specific protein in a biological sample from a patient or a purification or extract of a biological sample or a dilution thereof. In methods of the invention, quantifying may be performed by measuring the concentration of the analyte biomarker in the sample or samples. Biological samples that may be tested in a method of the invention include cerebrospinal fluid (CSF), whole blood, blood serum, plasma, urine, saliva, or other bodily fluid (stool, tear fluid, synovial fluid, sputum), breath, e.g. as condensed breath, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner.

Detection and/or quantification of analyte biomarkers may be performed by detection of the analyte biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (−TOF), MALDI (−TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing or monitoring according to the invention may comprise analysing a sample of cerebrospinal fluid (CSF) by SELDI TOF or MALDI TOF to detect the presence or level of the analyte biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the analyte biomarkers may be performed using an immunological method, involving an antibody, or a fragment thereof capable of specific binding to the analyte biomarker. Suitable immunological methods include sandwich immunoassays, such as sandwich ELISA, in which the detection of the analyte biomarkers is performed using two antibodies which recognize different epitopes on a analyte biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes that are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of analyte biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc.) is labelled with biotin and the other partner (surface, e.g. well, bead, sensor etc.) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for an analyte biomarker of the invention may be immobilised on an avidin or streptavidin surface, the immobilised ligand may then be exposed to a sample containing or suspected of containing the analyte biomarker in order to detect and/or quantify an analyte biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab′)₂ fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing or monitoring bipolar I or bipolar II disorders which comprises a biosensor, microanalytical, microengineered, nnicroseparation and/or immunochromatography system configured to detect and/or quantify any of the biomarkers defined herein.

The biomarker(s) of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Methods involving detection and/or quantification of one or more analyte biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-depressive disorder therapeutic; or the test substance can be novel synthetic or natural chemical entity, or a combination of two or more of the aforesaid substances.

There is provided a method of identifying a substance capable of promoting or suppressing the generation of the analyte biomarker in a subject, comprising exposing a test cell to a test substance and monitoring the level of the analyte biomarker within said test cell, or secreted by said test cell.

The test cell could be prokaryotic, however a eukaryotic cell will suitably be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing the analyte.

Screening methods also encompass a method of identifying a ligand capable of binding to the analyte biomarker according to the invention, comprising incubating a test substance in the presence of the analyte biomarker in conditions appropriate for binding, and detecting and/or quantifying binding of the analyte to said test substance.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of inhibiting, directly or indirectly, the activity of the analyte biomarker, or of suppressing generation of the analyte biomarker. The term “substances” includes substances that do not directly bind the analyte biomarker and directly modulate a function, but instead indirectly modulate a function of the analyte biomarker. Ligands are also included in the term substances; ligands of the invention (e.g. a natural or synthetic chemical compound, peptide, aptamer, oligonucleotide, antibody or antibody fragment) are capable of binding, suitably specific binding, to the analyte.

The invention further provides a substance according to the invention for use in the treatment of bipolar I or bipolar II disorders, or predisposition thereto.

Also provided is the use of a substance according to the invention in the treatment of bipolar I or bipolar II disorders, or predisposition thereto.

Also provided is the use of a substance according to the invention as a medicament.

Yet further provided is the use of a substance according to the invention in the manufacture of a medicament for the treatment of bipolar I or bipolar II disorders, or predisposition thereto.

A kit for diagnosing or monitoring bipolar I or bipolar II disorders, or predisposition thereto is provided. Suitably a kit according to the invention may contain one or more components selected from the group: a ligand specific for the analyte biomarker or a structural/shape mimic of the analyte biomarker, one or more controls, one or more reagents and one or more consumables; optionally together with instructions for use of the kit in accordance with any of the methods defined herein.

The identification of biomarkers for bipolar I or bipolar II disorders permits integration of diagnostic procedures and therapeutic regimes. Currently there are significant delays in determining effective treatment and hitherto it has not been possible to perform rapid assessment of drug response. Traditionally, many anti-depressant therapies have required treatment trials lasting weeks to months for a given therapeutic approach. Detection of an analyte biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized brain therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Furthermore, diagnostic biomarker tests are useful to identify family members or patients at high risk of developing bipolar I or bipolar II disorders. This permits initiation of appropriate therapy, or preventive measures, e.g. managing risk factors. These approaches are recognised to improve outcome and may prevent overt onset of the disorder.

Biomarker monitoring methods, biosensors and kits are also vital as patient monitoring tools, to enable the physician to determine whether relapse is due to worsening of the disorder, poor patient compliance or substance abuse. If pharmacological treatment is assessed to be inadequate, then therapy can be reinstated or increased; a change in therapy can be given if appropriate. As the biomarkers are sensitive to the state of the disorder, they provide an indication of the impact of drug therapy or of substance abuse.

The following studies illustrate the invention.

### EXAMPLE 1

### Identification of Bipolar Disorder Markers

This study measured levels of 247 molecules in serum collected from 32 Bipolar disorder (BD) patients and 32 well matched controls. Levels of all molecular analytes were determined using a highly reproducible multiplexed immunoassay platform. The correlation structure between all analytes was assessed to infer potential co-regulation structures.

A panel of 42 markers was found to be significantly altered in the BD group. Abnormalities in 30 of these markers remained significant after adjustment for all recorded baseline characteristics including age, sex, body mass index, smoking and cannabis consumption. Among the significant markers, a highly prominent correlation structure was found.

Methodology

Patients

In the present study, samples were investigated from patients suffering from Bipolar Disorder (BD) (n=32) and well matched controls (n=32). The ethical committees of the medical faculties of the partner universities approved the protocols of this study. Informed consent was given in writing by all participants and clinical investigations were conducted according to the principles expressed in the Declaration of Helsinki.

Sample Preparation

Blood was collected in S-Monovette 7.5 mL serum tubes (Sarstedt), incubated at room temperature for 2 hours to allow for blood coagulation and then centrifuged at 4000×g for 5 minutes. The supernatant was stored at −80° C. in Low Binding Eppendorf tubes.

Assay Methods

A total of 247 analytes were measured using a set of proprietary multiplexed immunoassays (Human MAP) at RBM in their Luminex-based, CLIA-certified laboratory (however measurement could equally be performed using singleton ELISA). Each antigen assay was calibrated using 8-point standard curves and performed in duplicate, and raw intensity measurements were interpreted into final protein concentrations using RBM's proprietary software. Machine performance was verified using quality control samples at low, medium, and high levels for each analyte in duplicate. All standard and quality control samples were in a complex plasma-based matrix to match the sample background. The autoimmune and infectious disease assays were qualitative and the results obtained for unknown samples were compared with established cut-off values. Because sera were analyzed at a previously optimized dilution, any sample exceeding the maximum concentration of the calibration curve was arbitrarily assigned the concentration of the highest standard, whereas those assayed below the minimum concentration of the calibration curve were assigned the value 0.0. For analysis, samples were ordered in a manner to avoid any sequential bias due to the presence or absence of disease, patient age, or age of serum sample. The analysis has been performed blind with regards to the diagnosis information.

Statistical Analysis

Differences between the disease and control group were determined by means of Analysis Of Variance (ANOVA). Recorded clinical baseline characteristics (sex, age, bmi, smoking and cannabis consumption) were accounted for if the interaction between the covariate and the diagnosis was not significant. The False Discovery Rate (FDR) was controlled according to Benjamini et al. (J Roy Statist Soc Ser B. 1995; 57:289-300).

Results

This study investigated levels of 247 molecular analytes in serum from 32 patients suffering from bipolar disorder and well matched controls (n=32). Demographic details can be found in Table 2:

TABLE 2
Demographic details of patients and healthy volunteers

| | Healthy | |
|---|---|---|
| | Controls | Bipolar |
| | (MDD) | Disorder |
| Number | 32 | 32 |
| Sex [m/f] | 10/22 | 13/19 |
| Age[years] | 32.9 ± 6.8 | 33.7 ± 10.4 |
| BMI | 23.7 ± 3.8 | 24.6 ± 3.7 |

Applying ANOVA, levels of 42 analytes were found to be significantly altered between the disease and the control group (Table 3). Thirty of these analytes were found not to feature a significant interaction between any of the recorded baseline characteristics and the diagnosis. Therefore, Analysis Of Covariance (ANCOVA) was applied to determine the difference between the disease and control group whilst accounting for the variability caused by the clinical baseline characteristics. Adjustment for multiple comparisons yielded q-values ranging from 0 to 0.31.

TABLE 3
Summary of significant findings
No interaction between diagnosis and covariates

| | | | fold |
|---|---|---|---|
| Analyte | p value | q value | change |
| Endothelin 1 | 1.57E−08 | 2.94E−06 | 3.61014 |
| Eotaxin-3 | 8.32E−08 | 7.82E−06 | 0.203872 |
| MIF | 1.19E−06 | 7.43E−05 | 3.225265 |
| HCC 4 | 0.00107 | 0.05027 | 1.393746 |
| LH (Luteinizing Hormone) | 0.003205 | 0.104625 | 1.879816 |
| Histone Antibody | 0.003339 | 0.104625 | 1.27791 |
| Histone H2b Antibody | 0.007973 | 0.1877 | 1.235616 |
| TNF alpha | 0.007987 | 0.1877 | 0.816594 |
| Apolipoprotein CIII | 0.010697 | 0.197814 | 0.848677 |
| IGF BP 2 | 0.011016 | 0.197814 | 1.31264 |
| Histone H4 Antibody | 0.011574 | 0.197814 | 0.88203 |
| Progesterone | 0.014283 | 0.207771 | 1.139588 |
| Anti Nuclear Antibody | 0.014714 | 0.207771 | 1.205537 |
| Testosterone | 0.015472 | 0.207771 | 1.204193 |
| Fas Ligand | 0.019248 | 0.218483 | 1.078418 |
| IgA | 0.020659 | 0.218483 | 0.809421 |
| C. trachomatis | 0.021061 | 0.218483 | 1.152246 |
| IgM | 0.021712 | 0.218483 | 0.740336 |
| IL-13 | 0.02212 | 0.218483 | 0.910708 |
| Histone H1 Antibody | 0.023936 | 0.218483 | 1.345398 |
| CTGF (Connective Tissue Growth | 0.025124 | 0.218483 | 1.109212 |
| Factor) | | | |
| Apolipoprotein A1 | 0.027828 | 0.218483 | 0.81619 |
| CD40 Ligand | 0.02801 | 0.218483 | 1.17977 |
| EGF | 0.028749 | 0.218483 | 1.487719 |
| CD40 | 0.029054 | 0.218483 | 1.18768 |
| Stem Cell Factor | 0.032319 | 0.23369 | 0.894831 |
| Lymphotactin | 0.035807 | 0.249324 | 8.195122 |
| C. jejuni | 0.04015 | 0.269576 | 1.259981 |
| T. cruzi | 0.045222 | 0.293165 | 1.231481 |
| GST | 0.048693 | 0.305141 | 1.087861 |

Interaction between diagnosis and covariates

| Analyte | pvalue | fold change |
|---|---|---|
| Alpha 2 Macroglobulin | 7.34E−07 | 0.795155 |
| ANG 2 Angiopoietin 2 | 6.58E−06 | 1.417331 |
| Thrombopoietin | 7.46E−05 | 0.716006 |
| Myeloperoxidase | 0.0003975 | 1.546075 |
| IL-8 | 0.0004004 | 4.07646 |
| TSP 1 | 0.0004574 | 1.115412 |
| IL-16 | 0.0006009 | 1.350993 |
| FSH (Follicle Stimulating | 0.0018785 | 2.724497 |
| Hormone) | | |
| MIP 1 beta | 0.0037124 | 1.342561 |
| SOD | 0.0081358 | 1.369085 |
| Fas | 0.0089503 | 1.289216 |
| Glucagon | 0.0471335 | N/A |

The serum levels of the molecules identified in this study appear to be sensitive and are likely to be specific predictors for the presence of bipolar disorder.

### EXAMPLE 2

### Identification of Further Bipolar Disorder Markers

187 analytes were analysed in accordance with the protocol described in International Patent Application No. PCT/GB2008/004186 which included 2 separate cohorts (Cohorts 1 and 6), the details of which are as follows:

Cohort 1 is exactly as described in International Patent Application No. PCT/GB2008/004186 and is from the Universities of Cologne, Muenster and Magdeburg. Cohort 1 was also used to obtain the biomarkers described in Example 1 hereinbefore.

Cohort 6 is a cohort from the US military. It consists of bipolar disorder patients and controls with full demographic details shown in Table 4:

TABLE 4
Demographic Details of Cohort 6

| Cohort 6 | Bipolar Disorder | Controls |
|---|---|---|
| Number | 110 | 110 |
| Sex (Male/Female) | 70/40 | 70/40 |
| Age | 21.3 ± 3.6 | 21.2 ± 3.5 |

The results of this study identified 21 biomarkers which demonstrated a sensitive and specific diagnostic for bipolar disorder (see Table 5).

TABLE 5
Summary of significant findings

| | | | Fold |
|---|---|---|---|
| Analyte | Centre where changed | p value | Change |
| Apolipoprotein A2 | 6 | <0.001 | 1.19 |
| Apolipoprotein B | 6 | <0.001 | 0.76 |
| Apolipoprotein CI | 6 | <0.001 | 1.24 |
| IL-11 | 6 | <0.004 | 0.77 |
| IL-17 | 6 | <0.001 | 1.17 |
| Thrombopoietin | 6 | <0.001 | 0.86 |
| Calbindin | 6 | 0.001 | 1.23 |
| Cancer Antigen 125 | 6 | 0.004 | 0.74 |
| CD5L | 6 | 0.010 | 0.89 |
| EGF receptor | 6 | <0.001 | 1.13 |
| FSH | 6 | 0.050 | 0.22 |
| IgM | 6 | 0.047 | 1.13 |
| IL-6 receptor | 6 | 0.010 | 1.12 |
| IL-7 | 6 | 0.011 | 0.82 |
| KIM-1 | 6 | 0.014 | 1.65 |
| MCP-2 | 6 | <0.001 | 1.22 |
| MMP-2 | 6 | 0.050 | 1.40 |
| PYY | 6 | <0.001 | 0.71 |
| TSH | 6 | 0.001 | 0.77 |
| Transferrin | 6 | <0.001 | 0.85 |
| Vitronectin | 6 | 0.002 | 0.95 |

### EXAMPLE 3

### Analysis of Bipolar Disorder Markers in Manic Psychosis Patients

The analytes identified in Examples 1 and 2 were analysed in plasma obtained from 29 patients diagnosed with a manic psychosis episode of bipolar disorder. This analysis was conducted in an analogous manner to the protocol described in Example 1.

The cohort used in this study (referred to as Cohort 10) is a cohort from Sheppard Pratt hospital (Baltimore, USA). The cohort consists of bipolar disorder patients undergoing manic psychosis along with control patients, the full demographic details are shown in Table 6:

TABLE 6
Demographic Details of Cohort 10

| Cohort 10 | Manic Psychosis | Controls |
|---|---|---|
| Number | 29 | 18 |
| Sex (Male/Female) | 10/19 | 6/12 |
| Age | 32 ± 10 | 34 ± 15 |

The results of this study identified the biomarkers listed in Tables 3 and 5 which are likely to provide a sensitive and specific diagnostic for bipolar disorder and also the manic psychosis episode of bipolar disorder (see Table 7).

TABLE 7
Results of Manic Psychosis Analysis

| | | fold |
|---|---|---|
| Analyte | p value | change |
| Cancer Antigen 125 | 0.037001 | 1.739062 |
| MIF | 0.007109 | 2.770678 |
| TNF-alpha | 0.020836 | 2.604048 |
| Progesterone | 0.000148 | 2.193754 |
| CD40 Ligand | 0.002005 | 3.453537 |
| EGF | 0.004679 | 5.135547 |
| CD40 | 0.000973 | 1.845618 |
| Alpha-2 Macroglobulin | 0.015733 | 1.674943 |
| Thrombopoietin | 0.000917 | 1.623774 |
| Myeloperoxidase | 0.001518 | 8.516738 |
| IL-16 | 0.008646 | 2.015265 |
| MIP-1beta | 0.022203 | 4.925972 |
| FAS | 0.031917 | 1.329775 |
| Calbindin | 0.04042 | 1.225588 |

1. A method for using one or more first analytes selected from the group consisting of: Cancer Antigen 125, HCC-4, Apolipoprotein B, IL-11, CD5L, IL-6 receptor, Kidney injury molecule 1 (KIM-1), MMP-2, Transferrin, Testosterone, C. jejuni, T. cruzi and Glucagon, as a biomarker for bipolar I or bipolar II disorders, or predisposition thereto.

2. The method of claim 1, wherein the first analyte biomarker is Cancer Antigen 125.

3. The method of claim 1, additionally comprising the use of one or more second analyte biomarkers selected from the group consisting of Eotaxin-3, LH (Luteinizing Hormone), Histone H2b Antibody, Apolipoprotein CIII, Histone H4 Antibody, Fas Ligand, IgA, C. trachomatis, IL-13, Histone H1 Antibody, CTGF (Connective Tissue Growth Factor), CD40 Ligand, EGF, Stem Cell Factor, Lymphotactin, Myeloperoxidase, TSP 1, IL-16, MIP-1 beta, Apolipoprotein A2, Apolipoprotein CI, IL-17, Thrombopoietin, Calbindin, EGF receptor, Follicle Stimulating Hormone (FSH), ANG 2 (Angiopoietin 2), IL-7, MCP-2, Peptide YY (PYY), Thyroid Stimulating Hormone (TSH; beta subunit), Vitronectin, Endothelin 1, MIF, Histone Antibody, TNF alpha, IGF BP 2, Progesterone, Anti Nuclear Antibody, IgM, Apolipoprotein A1, CD40, GST, Alpha 2 Macroglobulin, IL-8, SOD and Fas.

4. (canceled)

5. (canceled)

6. (canceled)

7. (canceled)

8. The method of claim 1, wherein one or more of the biomarkers may be replaced by a molecule, or a measurable fragment of the molecule, found upstream or downstream of the biomarker in a biological pathway.

9. A method of diagnosing bipolar I or bipolar II disorders, or predisposition in an individual thereto, comprising:
(a) obtaining a biological sample from an individual;
(b) quantifying the amounts of the analyte biomarkers as defined in any of claims 1 to 7;
(c) comparing the amounts of the analyte biomarkers in the biological sample with the amounts present in a normal control biological sample from a normal subject, such that a difference in the level of the analyte biomarkers in the biological sample is indicative of bipolar I or bipolar II disorders, or predisposition thereto.

10. A method of monitoring efficacy of a therapy in a subject having, suspected of having, or of being predisposed to bipolar I or bipolar II disorders, comprising detecting and/or quantifying, in a sample from said subject, the analyte biomarkers as defined in claim 1.

11. The method as defined in claim 9, which is conducted on samples taken on two or more occasions from a test subject.

12. (canceled)

13. (canceled)

14. (canceled)

15. The method of claim 9, further comprising comparing the amount of the biomarker present in said test sample with one or more controls.

16. The method as defined in claim 15, comprising comparing the amount of the biomarker in a test sample with the amount of the biomarker present in a sample from a normal subject.

17. The method of claim 9, wherein samples are taken prior to and/or during and/or following therapy for bipolar I or bipolar II disorders.

18. The method of claim 9, wherein samples are taken at intervals over the remaining life, or a part thereof, of a subject.

19. The method of claim 9, wherein quantifying is performed by measuring the concentration of the analyte biomarker in the or each sample.

20. The method of claim 9, wherein detecting and/or quantifying is performed by one or more methods selected from SELDI (−TOF), MALDI (−TOF), a 1-D gel-based analysis, a 2-D gel-based analysis, Mass spec (MS), reverse phase (RP) LC, size permeation (gel filtration), ion exchange, affinity, HPLC, UPLC or other LC or LC-MS-based technique.

21. The method of claim 9, wherein detecting and/or quantifying is performed using an immunological method.

22. The method of claim 9, wherein the detecting and/or quantifying is performed using a biosensor or a microanalytical, microengineered, microseparation or immunochromatography system.

23. (canceled)

24. (canceled)

25. (canceled)
